(19) United States
(12) Patent Application Publication
(10) Pub. No.: US 2021/0317445 A1
(43) Pub. Date: Oct. 14, 2021

(54) SYSTEM AND METHOD FOR GENE EDITING CASSETTE DESIGN

(71) Applicant: Inscripta, Inc., Boulder, CO (US)

(72) Inventors: Andrea HALWEG-EDWARDS, Boulder, CO (US); Joshua SHORENSTEIN, Boulder, CO (US); Andrew GARST, Boulder, CO (US); Craig STRUBLE, Pleasanton, CA (US); Miles GANDER, Boulder, CO (US); Juhan KIM, Pleasanton, CA (US); Bryan LELAND, Boulder, CO (US); Eileen SPINDLER, Boulder, CO (US); Paul HARDENBOL, Pleasanton, CA (US)

(21) Appl. No.: 16/945,575

(22) Filed: Jul. 31, 2020

Related U.S. Application Data
(63) Continuation of application No. 16/903,324, filed on Jun. 16, 2020.
(60) Provisional application No. 63/007,266, filed on Apr. 8, 2020.

(51) Int. Cl. C12N 15/10

(52) U.S. Cl. CPC C12N 15/1089

(57) ABSTRACT

The present disclosure is drawn to creating cassette designs for nucleic acid-guided nuclease editing. In designing editing cassettes, a set of edit specifications must first be obtained. These edit specifications are taken together with a set of configuration parameters to start a computational pipeline that generates a collection of cassette designs. The process of designing editing cassettes involves the following exemplary steps: 1) creation of a set of candidate cassette designs for each unique edit specification, 2) enumeration of features describing biophysical characteristics of each candidate design, 3) providing each candidate design with a score, and 4) returning a number of scored and rank-ordered candidate cassette designs for each edit specification.

### CROSS-REFERENCE TO RELATED APPLICATIONS

This application is a continuation of U.S. patent application Ser. No. 16/903,234, filed Jun. 16, 2020, which claims benefit of United States provisional patent application Ser. No. 63,007,266, filed Apr. 8, 2020, which are herein incorporated by reference.

### BACKGROUND

### Field

Embodiments of the present disclosure generally relate to gene editing, and more particularly to methods and systems for the creation of editing cassettes, and pools of editing cassettes, for performing nucleic acid-guided nuclease editing.

### Description of the Related Art

Gene editing has become an important part of research in medicine, biology, and a host of other areas of endeavor. A relatively new discovery, CRISPR-enabled DNA editing, has revolutionized the gene-editing field. Specifically, it is possible to generate tens of thousands of programmed edits in a cell population by leveraging CRISPR endonuclease specificity and homology-directed repair. To edit a gene, a guide RNA (gRNA) and donor DNA are simultaneously introduced into a live cell. The gRNA and CRISPR endonuclease form a macromolecular complex, which will interact with a target site in the genome, extrachromosomal vector, or other editable component of a live cell, catalyzing a cut on the cellular sequence (e.g. “double-strand break” or “single-strand nick”). The cell then repairs the cut DNA, and one mechanism of DNA-repair is via homologous recombination. Cut DNA that is repaired with donor DNA results in an edited gene sequence. By manipulating a nucleotide sequence of the gRNA, the nucleic acid-guided endonuclease may be programmed to target any DNA sequence as long as an appropriate protospacer adjacent motif (PAM) is present.

In prior approaches, researchers introduced pools of gRNAs and pools of donor DNAs separately into a population of cells. However, in addition to being expensive and time-consuming, this process does not scale well for creating large diverse populations of edited cells.

More recently, gene-editing cassettes have been created that include the gRNA covalently-linked to a donor DNA repair template; thus, every cell that receives a vector containing an “editing cassette” automatically receives both nucleic acids necessary to carry out editing. In creating these cassettes, a number of criteria need to be taken into consideration to produce a pool of diverse editing cassettes targeting hundreds to tens of thousands, and more, editable sites of a cellular genome.

What is needed are methods and systems for creating pools of diverse editing cassettes designs for performing genome editing of up to hundreds of thousands of genetic loci in a population of live cells in a single editing round. The present disclosure provides such methods and systems.

### SUMMARY

The systems and methods of the disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure provide advantages that include the development of gene-editing cassette designs, and pools of such designs.

Certain aspects of the present disclosure provide a system for designing a gene editing cassette that includes a design library specification comprising an edit description and a target sequence, and a candidate cassette design engine that receives the design library specification as input and modifies the target sequence with the edit description to produce a candidate cassette design comprising a cassette design sequence.

Certain aspects of the present disclosure provide a method for designing a gene editing cassette that includes parsing a design library specification to identify a target sequence comprising a PAM-protospacer, an endonuclease capable of cleaving the target sequence, and an edit description, modifying the target sequence with the edit description to generate a modified target sequence, generating a homology arm comprising the modified target sequence, assembling a candidate cassette design comprising the homology arm, and returning the candidate cassette design.

Certain aspects of the present disclosure provide a non-transitory computer-readable medium comprising instructions that, when executed by a processor of a processing system, cause the processing system to perform a method for designing a gene editing cassette, the method including parsing a design library specification to identify a target sequence comprising a PAM-protospacer, an endonuclease capable of cleaving the target sequence, and an edit description, modifying the target sequence with the edit description to generate a modified target sequence, generating a homology arm comprising the modified target sequence, assembling a candidate cassette design comprising the homology arm, and returning the candidate cassette design.

Certain aspects of the present disclosure provide a processing system including memory comprising computer-executable instructions, a processor configured to execute the computer-executable instructions and cause the processing system to perform a method for designing a gene editing cassette, the method including parsing a design library specification to identify a target sequence comprising a PAM-protospacer, an endonuclease capable of cleaving the target sequence, and an edit description, modifying the target sequence with the edit description to generate a modified target sequence, generating a homology arm comprising the modified target sequence, assembling a candidate cassette design comprising the homology arm, and returning the candidate cassette design.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 depicts a system for designing gene editing cassettes and cassette pools according to an embodiment.

FIG. 2 depicts a design library specification for editing cassette designs according to an embodiment.

FIG. 3 depicts a design library configuration parser, a candidate design feature builder, a candidate design score calculator, and a rank-ordered candidate design library of the system for designing editing cassettes and cassette pools, according to an embodiment.

FIG. 4 depicts a candidate cassette design engine of the system of designing editing cassettes and cassette pools, according to an embodiment.

FIG. 5 depicts a method for initializing an editing cassette design according to an embodiment.

FIG. 6 depicts a method for scoring cassette designs according to an embodiment.

FIG. 7 depicts a method for generating an editing cassette design according to disclosed embodiments.

FIG. 8 a method to determine if an endonuclease will cleave a PAM protospacer of a cassette design, according to disclosed embodiments.

FIG. 9 depicts data illustrating edit efficiency boost using the intervening edit strategy according to embodiments of systems and methods disclosed herein.

FIG. 10 depicts data illustrating genomic edits from design libraries created by embodiments of systems and methods disclosed herein.

FIG. 11 depicts an exemplary method for generating an editing cassette design, according to embodiments.

FIG. 12 depicts an exemplary processing system for generating an editing cassette design, according to embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In the following, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

Aspects of the present disclosure provide apparatuses, methods, processing systems, and computer-readable mediums for developing a DNA-editing cassette design, or pool(s) of cassette designs.

CRISPR (Clustered Regularly Interspaced Short Palindromic Repeats) technology is a simple yet powerful tool for editing genomes (i.e., genetic material) in live cells. CRISPR gene editing technology allows researchers to alter DNA sequences and thus modify gene function. CRISPR technology was adapted from the natural defense mechanisms of bacteria and archaea. These organisms use CRISPR-derived nucleic acids and specialized enzymes to foil attacks by viruses and other foreign bodies. This defense is accomplished primarily by chopping up and destroying the DNA of the foreign invader. However, when engineered CRISPR components are transferred to other organisms, it allows for the modification of genes or “gene editing” in these other organisms.

Researchers in academia and industry seek to edit gene sequences for a variety of reasons. Among these are the development of therapies to treat or prevent disease, growing organs for transplant, mitigating the effects of aging, developing organisms able to produce bio-fuels, pharmaceuticals or other resources, increasing crop yields, as well as a growing list of industrial and research applications that are discovered as genetic sequences, and their effects, are better understood.

In order to edit a gene sequence, several components must interact with the targeted DNA at an intended edit site. These components include, and are not limited to, the ribonucleoprotein complex formed between a guide RNA (gRNA), a nucleic acid-guided endonuclease (examples include: Cas9, Cas12/Cpfl, MAD2, MAD7, other MADzymes, or other nucleic acid-guided endonucleases now known or later developed), and a repair template (sometimes referred to as a “donor DNA,” “donor sequence,” or “homology arm”). In prior approaches, gRNAs and repair templates were introduced as separate molecules. However, it has been demonstrated that if efficient genome editing in multiplex (e.g. “in parallel”) is desired, then providing a complex comprising a covalent linkage of the gRNA and the repair template, and potentially additional molecules, provides more predictable outcomes. This complex sometimes referred to as a “cassette” or “editing cassette.” This covalently-linked group of molecules enables the generation of complex pools of editing cassette designs useful for editing hundreds, thousands, tens of thousands or even hundreds of thousands and more, loci in a cell population, in a “one-pot” reaction.

The covalently linked gRNA and repair template is one form of an “editing cassette.” When an editing cassette is inserted into a cloning vector backbone (a DNA sequence that can be stably maintained in an organism), an “editing vector” is formed. Every cell that receives an editing vector automatically receives both nucleic acids (e.g., gRNA and repair template) necessary to carry out editing. For descriptions of editing cassettes, see, e.g., U.S. Pat. Nos. 10,240,499; 10,266,849; 9,982,278; 10,351,877; 10,364,442; 10,435,715; and 10,465,207, and U.S. patent application Ser. No. 16/550,092, filed 23 Aug. 2019; and U.S. Pat. No. 16,551,517, filed 26 Aug. 2019, all of which are incorporated by reference herein.

As used herein, a “cassette” or “editing cassette” is a generic term to describe a DNA sequence that can be cloned into an extrachromosomal vector backbone. An editing cassette encodes 1) one or more guide RNA (gRNA) sequences designed to specifically target particular region(s) of a “target DNA” (or “target sequence” or “target genome”) within a cell of interest; 2) a repair template that is used to repair the cut target DNA, and in some embodiments there may be additional molecules complexed with the gRNA and repair template; and 3) other functional elements described in more detail below. The repair template may repair the cut site using homology-directed repair or an alternative mechanism depending on the repair template design and the nature of the CRISPR endonuclease and/or repair functionality made available to the cell at the time of DNA editing.

The term “target DNA” is used to describe any DNA sequence (genomic or otherwise) that is targeted for editing by the expressed RNA-guided nuclease in complex with the gRNA. In addition to the editing cassette, the extrachromosomal vector backbone typically comprises additional genetic elements such as one or more nuclear localization sequences with a promoter driving transcription thereof; transcription terminator elements; a promoter driving an antibiotic resistance gene; one or more origins of replication and other genetic elements known to those of ordinary skill in the art. As used herein, a “gRNA” is a term to describe the RNA molecule that forms a ribonucleoprotein complex with the CRISPR endonuclease. This gRNA is comprised of two functional sections, herein referred to as the “CR” (or “crRNA” or “crRNA repeat” or “crRNA scaffold”) and “SR” (“protospacer-complementary sequence” or “target-binding sequence” or “tracrRNA guide segment” or “crRNA spacer region” or “spacer sequence”) cassette components.

Aside from the gRNA and the repair template, other functional components of an editing cassette may include and are not limited to, amplification primer binding sites (“amplification” means using a polymerase chain reaction (PCR) to produce many copies of a DNA molecule to facilitate operational use of this material), regulatory elements for gRNA expression (including and not limited to promoter or terminator sequences), restriction enzyme recognition sequences, and identification markers called “barcodes”.

In the context of a gene-editing cassette, each functional component can be considered “modular”, meaning that functional components of an editing cassette may be in any order specified by a designer. This flexibility allows cassette designers to test addition, subtraction, modification, and rearrangement of functional components of their designs, enabling users to rapidly test different cassette design architectures (where “architecture” describes an arrangement of functional components) in order to discover optimal cassette design structure. Moreover, when a particular cassette architecture has been determined to be optimal, this architecture can be set in a cassette design system described herein, such that it will be selected as a default or selectable setting, given the user's specified editing organism (strain or cell type) and the editing kit (examples include and are not limited to single editing kit and combinatorial editing kit).

While the systems and methods described herein are agnostic to the cassette architecture, one with ordinary skill given the teachings of the present disclosure will understand that the arrangement of functional components can have a profound effect on the efficacy of an editing cassette design. For example, the order of a crRNA repeat (a “CR” component, discussed above and further below) and crRNA spacer region (“SR”) is dependent on the CRISPR system used. For example, if a Type V CRISPR system (e.g., MAD7) is used, then the crRNA repeat element must precede the spacer sequence in order, within the cassette. As another example, if a Type II CRISPR system (e.g., Cas9) is used, the spacer sequence must precede the crRNA repeat element in order, within the cassette.

Each cassette typically targets two edit regions: an “intended edit”, which represents the set of edits that a user wishes to introduce into the target DNA, and an ancillary edit (sometimes referred to as an “auxiliary edit”), which is a set of one or more swap edits that are predicted to increase the cassette design's potential to result in complete incorporation of both edit regions (i.e., intended and ancillary) into the target DNA following an editing event. In some embodiments, insertion and/or deletion edits may be used in addition to/instead of swap edits, when implementing an ancillary edit. Ancillary edits may edit a PAM and/or protospacer sequence in order to block the endonuclease-gRNA complex from cutting the edited sequence beyond the intended edit. Ancillary edits that modify the PAM and/or protospacer sequence effectively “immunizes” the edited sequence against further cutting by the particular endonuclease used in the previous edits. Ancillary edits can over-write the PAM or the protospacer or both. Optionally, ancillary edits may also be encoded in the region between the “intended edit” region and a nuclease cut site, bolstering the cut repair efficiency. To the extent possible, care is taken during the cassette design process to confer ancillary edits that are biologically inert; that is, they are designed in an effort to optimize avoidance of collateral damage to the cell. Specifically, if edits are being made within a “coding region”, or codon, of a gene (i.e., a region either naturally or synthetically designed to produce a particular protein, amino acid, or other substance), the cassette design process defaults to encoding ancillary edits as synonymous codon changes, ensuring the amino acid, protein, or other substance for which the coding region is designed to produce, is the same as the unedited sequence of the coding region.

In contrast to ancillary edits, which may be “swap” mutations in some embodiments and include insertion and/or deletion edits in other embodiments, the end-user's intended edit can fall into one of four general categories: deletion, insertion, swap, and replacement. A deletion mutation modifies the target DNA by removing nucleotides, or “base-pairs” if the double-stranded product is considered, resulting in a DNA sequence that is shorter than the unedited DNA sequence. An insertion mutation is the result of adding nucleotides or base-pairs to the target DNA during the editing process, thereby creating an edited DNA sequence that is longer than the unedited DNA sequence. A swap mutation results in a DNA sequence that is the same length as the unedited DNA sequence and contains one or more nucleotide or base-pair changes. A replacement is the combination of removing nucleotides from the target DNA and simultaneously inserting new nucleotides, resulting in an edited sequence that may be shorter, longer, or the same size as the unedited sequence.

The methods and systems used to provide the instructions to design editing cassettes and pools of editing cassettes (or “design libraries”) are the subject of the present disclosure. Developing editing cassette designs (i.e. instructions to synthesize cassettes containing at least the above-described cassette components), and design libraries, according to customer needs requires consideration of a large number of parameters that will influence a given design as well as redundant alternatives (i.e. design versions that are functionally equivalent but incorporate different nucleotide sequences) to the design. For example, cell type (for mammalian systems), cell strain (for microbial systems), the sequence being edited, the positional coordinate(s) of the intended edit region, the desired edit sequence, the desired CRISPR endonuclease that will be used during editing, relative PAM-dependent cut activity for the specified nuclease, whether to allow incorporation of ancillary edits, optimization of the distance between the CRISPR endonuclease cut site and the user's intended edit, that collectively represent a “Cassette Design Architecture,” as well as which sequences to consider when searching for off-target effects. One of skill in the art given the teachings of the present disclosure will appreciate the variety of additional parameters available when designing an editing cassette.

In order to create an individual cassette design or a collection thereof or pool of individual cassette designs, a set of corresponding edit specifications must be obtained from the customer or other end-user. These edit specifications are taken together with a set of default configuration parameters to start a computational pipeline that generates a collection of cassette designs. The process of designing editing cassettes involves the following exemplary steps: 1) creation of a set of candidate cassette designs for each unique edit specification, 2) enumeration of features describing biophysical characteristics of each candidate design and/or creation of sequence embeddings or other abstract features such as those created from training a neural network, 3) providing each candidate design with a score, reflecting its relative potential to give rise to the complete intended edit event, and 4) returning the number of scored and rank-ordered candidate designs requested by the end-user for each edit specification. The elements of the cassette design pipeline are described below. The completed cassette design library may then be synthesized by a DNA oligomer manufacturing process (a process by which DNA sequences are translated into physical macromolecular polymers), inserted into one or more vector backbones, then, for example, provided to an automated multi-module cell processing system used to produce a library of cells comprising tens to hundreds of thousands of rationally-designed genome edits according to a customer request. Inscripta Inc. of Boulder Colo. has developed tabletop systems that automate gene editing in live cells, as described in U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,266, issued 5 Nov. 2019; U.S. Pat. No. 10,519,437 issued 31 Dec. 2019; U.S. Pat. No. 10,584,333, issued 10 Mar. 2020; U.S. Pat. No. 10,584,334, issued 10 Mar. 2020; and U.S. patent application Ser. No. 16/750,369, filed 23 Jan. 2020; Ser. No. 10/822,249, filed 18 Mar. 2020; and Ser. No. 16/837,985, filed 1 Apr. 2020, all of which are herein incorporated by reference in their entirety. The process of creating editing cassette pools described in the present disclosure may be used in these and other automated systems.

### Example Cassette Design Editing System

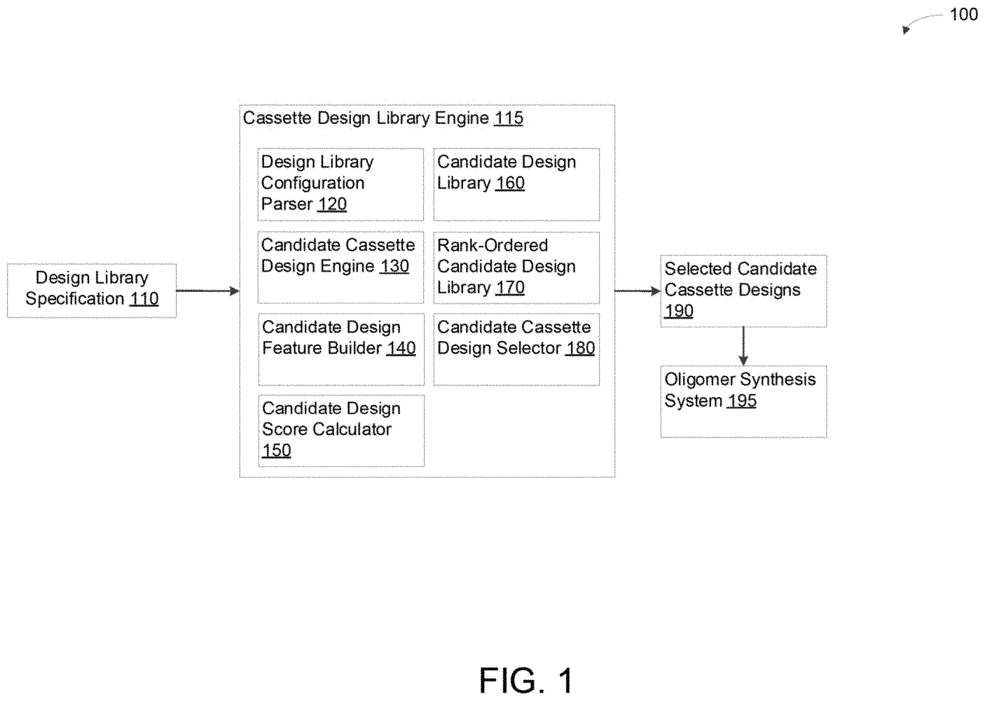

FIG. 1 depicts a system 100 for designing gene editing cassettes and cassette pools according to an embodiment.

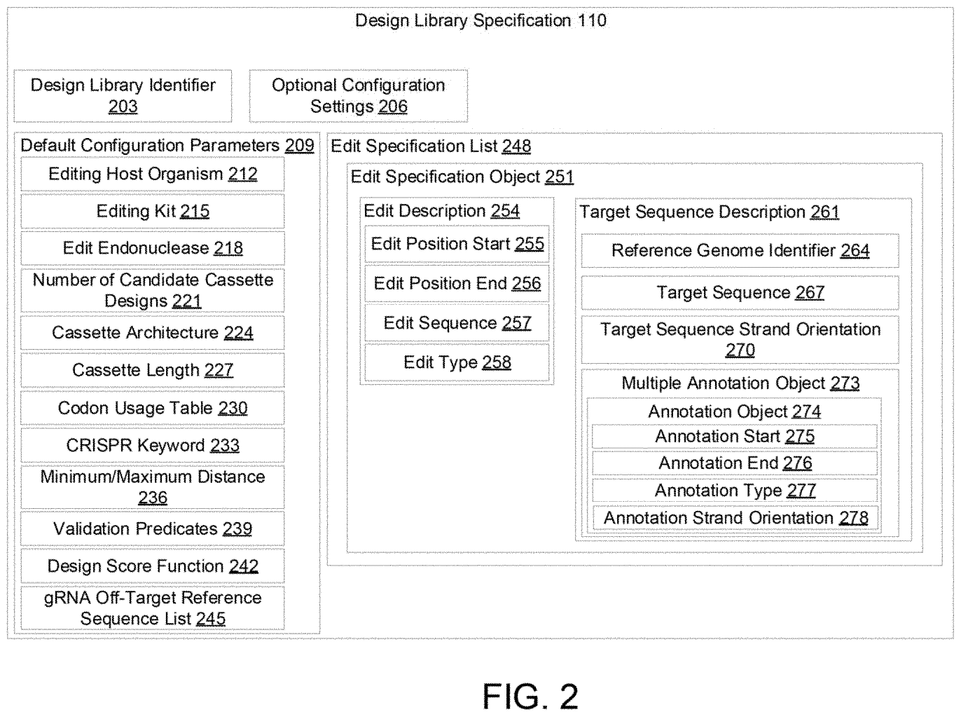

A gene-editing cassette design library engine 115 of system 100 takes as input a design library specification 110, described in detail below in connection with FIG. 2 that includes system configuration elements as well as end-user design elements for incorporation into a library of editing cassette designs. The cassette design library engine 115 includes a design library configuration parser 120 that parses the design library specification 110, and a candidate cassette design engine 103 that may produce one or more candidate cassette designs per edit specification object 251 of FIG. 2. It is understood by one of skill in the art that although certain elements of the disclosure reference objects, this does not limit any embodiment to an implementation with object-oriented programming languages, or the like. As is known, an object is a collection of data (i.e., data as such in various forms known to one of skill, such as strings, arrays, vectors, databases, files, etc.) and methods (i.e., computer-readable and computer-executable instructions), which can be considered together as an object per se in the context of object oriented programming, or as separate elements in procedural programming, while maintaining similar functionality and outcomes. Cassette design library engine 115 further includes a candidate design feature builder 140 that calculates a vector array for each candidate cassette sequence comprised of biophysical characteristics (including and not limited to the structural stability of subsequences of the gRNA) and summary statistics describing sequence composition of the cassette sequence (including and not limited to the GC sequence content of the cassette sequence). Cassette design library engine 115 includes a candidate design score calculator 150 that develops a design score for each editing cassette design in a candidate design library 160 produced by cassette design engine 103, a rank-ordered candidate design library 170 that is comprised of a rank-ordered set of editing cassette designs, and a candidate cassette design selector 180 that selects from the rank-ordered candidate design library a set of selected design candidate designs 190 to return to the end-user and provided to an oligomer synthesis system 195 for the fabrication of gene-editing cassettes. Embodiments of each of the foregoing components are described in further detail below.

FIG. 2 depicts a design library specification 110 for editing cassette designs according to an embodiment.

The design library specification 110 includes a design library identifier 203, and a set of optional design configuration settings 206 that an end-user is permitted to modify.

The design library specification 110 further includes a set of default configuration parameters 209 that are set by the unique combination of a user-specified editing kit 215 and the user-specified editing host organism 212 that describes a strain or cell type (e.g., E. coli MG1655, S. cerevisiae S288c, H. sapiens Hap1). The default configuration parameters include definitions for an edit endonuclease 218 (e.g., CAS9, MAD7) to be used in the editing process, comprising member variables that specify the location of a protospacer with respect to a PAM and the length of the protospacer-complementarity region required for optimal gRNA activity. Additionally, the design library specification 110 includes an edit specification list 248 typically provided by an end-user of the system 100, comprising one or more edit specification objects 251. Each edit specification object 251 is comprised of attributes/features of the edit sequences requested by the end-user.

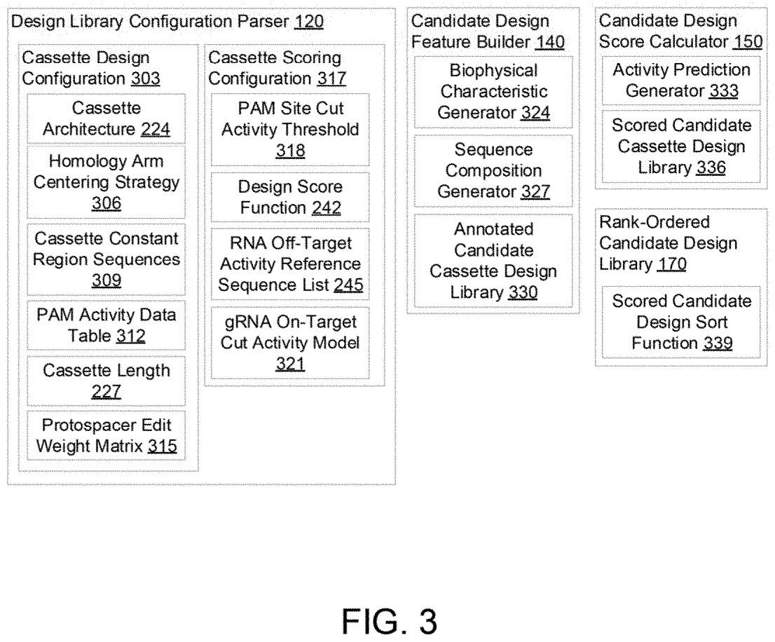

Many of the default configuration parameters 209 are established by system administrators and may be overridden by end-users through optional configuration settings 206, impacting editing cassette designs and the output of the cassette design library engine 115. Examples of default configuration parameters 209 include a number of candidate cassette designs 221 to return per unique edit specification object 251, a cassette architecture 224 of FIG. 3, a cassette length 227 that describes the complete length of the cassette under design, expressed in number of nucleotides, a codon usage table 230 utilized when selecting alternate codons for building ancillary edits, directives used to instantiate a homology arm generator object 460 (e.g., a cut repair template), a CRISPR keyword 233 used to instantiate a CRISPR system object 436, a minimum/maximum distance 236 allowed between the positional start of the user's intended edit site and a specified region of the PAM-protospacer motif, and a set of design validation predicates 239 used in a cassette validator object 424, all of which are described below. The default configuration parameters 209 also provide instructions for scoring each cassette design, with specifications for a cassette design score function 242, a gRNA off-target reference sequence list 245, and whether to include the reference genome assembly when searching for potential off-target gRNA binding sites (Boolean parameter not shown).

The edit specification list 248 is comprised of one or more edit specification objects 251. Each edit specification object 251 can result in 1) multiple redundant cassette designs, 2) a single cassette design, or 3) no cassette designs (e.g., if no cassette design resulting from a given edit specification object 251 was found to be viable). Each edit specification object 251 is associated with one or more edit descriptions 254 that include an edit position start 255 that defines a nucleotide position in a target sequence 267, an edit position end 256, and an edit sequence 257 intended by the user expressed as a sequence of nucleotides. The target sequence 267 defines the nucleotide sequence of the DNA of the editing host organism 212, of a given edit specification object 251, that an end-user intends to edit in a manner described by one or more edit description(s) 254. Collectively, the edit specification list 248 indicates one or more edit descriptions 254, each defined as an edit type 258 to be performed at the desired location, such as one of a swap, insertion, deletion, or substitution (e.g., replacement). The positional coordinates of edit position start 255 and edit position end 256, indicating the edit site can be referenced as absolute or relative nucleotide positions with respect to a reference genome, such as identified by a reference genome identifier 264 or a target sequence 267, respectively. There may be multiple sets of edit descriptions 254 associated with a single target sequence 267 of target sequence description 261.

Target sequence description 261 is a specification of the genome to be edited. This sequence includes the reference genome identifier 264 that identifies a discrete genome to be targeted for editing, the target sequence 267 of interest within the reference genome, and a target sequence strand orientation 270 that identifies a particular strand in the reference genome.

There are many options available for customers with regard to selecting a target sequence 267 and its associated annotation object 274 of a multiple annotation object 273. The target sequence 267 is a subsequence of the reference genome sequence associated with reference genome identifier 264. Customer options for target sequence 267 selection are limited only by customer design decisions based on customer needs.

The cassette design library engine 115 can work with any DNA sequence registered with the engine using the reference genome identifier 264. The engine can build editing cassette designs for any DNA sequence, whether occurring in nature, previously edited, partially sequenced, or partially synthesized, including genome sequences classified as Eukaryota (including fungi, mammals, and plants), Archaea, and Bacteria as well as that of viral genome assemblies.

Target sequence description 261 includes the multiple annotation object 273 in which each annotation object 274 is comprised of an annotation start 275 and annotation end 276, indicating positional coordinates for the annotated feature relative to the target sequence 267, an annotation type 277 indicating the biological activity of the annotated feature, and an annotation strand orientation 278 with respect to the target sequence 267. The annotation object 274 can describe any characteristic of the target sequence 267, including a particular gene sequence, a functional domain, or a splice site within the target sequence 267 where an edit is to be made. The target sequence description 261 also includes the target sequence strand orientation 270 that specifies the target sequence 267 orientation with respect to the reference genome identifier 264. There may be multiple edit descriptions 254 associated with a target sequence description 261 through the edit specification 251, signifying multiple edit sites within the target sequence 267 that are desired by the customer. The target sequence 267 typically includes “buffer” (or “flanking”) regions both upstream and downstream of the annotation boundaries surrounding the edit site, defined by one or more annotation start 275 and annotation end 276, respectively, of the target sequence 267. These left-flanking and right-flanking sequences are typically 100 nucleotides long, and in some embodiments, may be longer or shorter. The entire target nucleotide sequence 267 is sometimes referred to as a buffered nucleotide sequence.

FIG. 3 depicts the design library configuration parser 120, candidate design feature builder 140, candidate design score calculator 150, and a rank-ordered candidate design library 160, of the cassette design library engine 115.

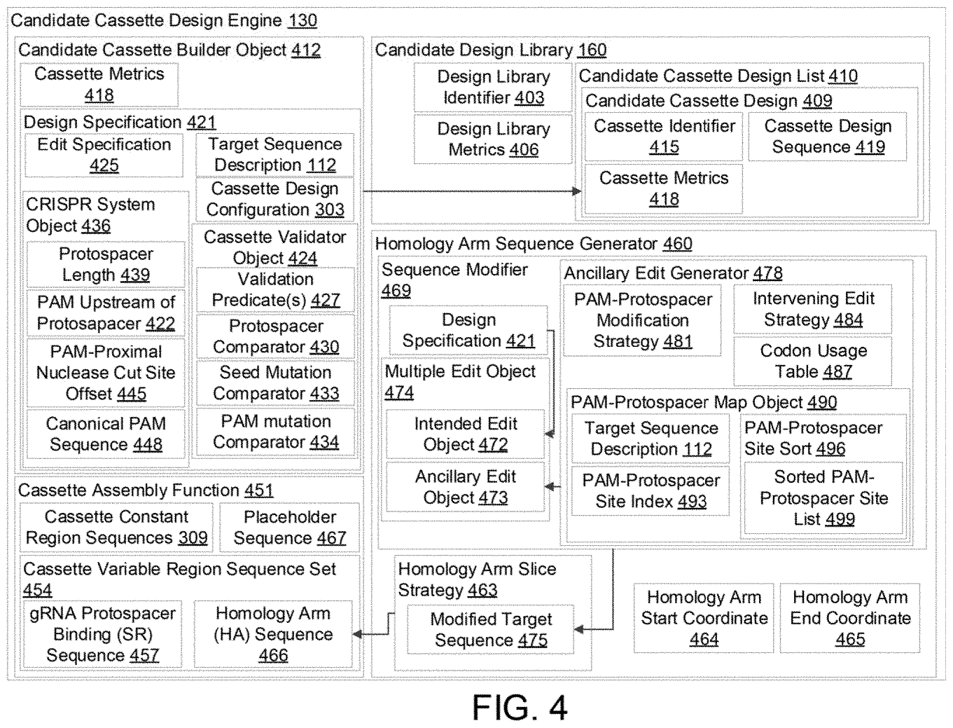

The design library specification 110 is an input of the design library configuration parser 120 that includes a cassette design configuration 303 and a cassette scoring configuration 317. Each of these components represent objects instantiated (e.g., create data structures and methods) by the design library configuration parser 120, and specify how to instantiate a candidate cassette builder object 412 (of FIG. 4) and the candidate design score calculator 150, which are used to build and score individual candidate cassette design(s) 409, respectively.

The candidate cassette design library engine 115 uses the cassette design configuration 303 along with a number of objects provided by the design library specification 110 as described in connection within FIG. 2, to instantiate the candidate cassette builder object 412 of FIG. 4. The cassette design configuration 303 defines settings used by the cassette builder object 412 to construct an editing cassette design. Settings encapsulated in the cassette design configuration 303, include and are not limited to, the cassette architecture 224, homology arm centering strategy 306, cassette constant region sequences 309, PAM activity data table 312, cassette length 227, and protospacer edit weight matrix 315. The cassette architecture 224 describes subsequences (i.e. components) of a cassette design, as well as the arrangement and order of those components that in one embodiment is represented as a set of two-letter codes. For example, the architecture string “SR_CR_HA” specifies that the “SR” sequence, representing the protospacer-complementarity region of the gRNA, precedes the “CR” sequence, representing the “crRNA” structural domain that binds to the CRISPR nuclease, and the cassette design terminates with the “HA” sequence, representing the homology arm used to repair and edit the target sequence. Homology arm centering strategy 306 contains a design specification declaring which sequence feature to place at the center of the homology arm repair template on a modified target sequence 475, described below in connection with FIG. 4. Depending upon user specifications, the homology arm may be centered on the edit sequence 257, while in other embodiments, the homology arm may be centered on a PAM motif, PAM-proximal cut site or a user-chosen region of the edit sequence 257. Homology arm centering strategy 306 is used by a homology arm sequence generator 460 (of FIG. 4) to determine a topology of a homology arm sequence, for example, that includes a homology arm start coordinate 464 and a homology arm end coordinate 465 with respect to the modified target sequence 475, among other elements.

The cassette constant region sequences 309 of the cassette design configuration 303 defines regions of the cassette architecture 224 that remain constant in terms of number and composition of nucleotides. PAM activity data table 312 specifies a data table containing PAM sequences, represented using IUPAC symbols and sequences for DNA nucleotides (e.g. ‘AAAA’ or ‘NRG’), and corresponding CRISPR nuclease cut activity for protospacer sequences adjacent to each PAM sequence. The protospacer edit weight matrix 315, a data table containing columns that represent protospacer positions and rows that represent nucleotide changes (e.g. A changed to G), specifies the efficiency with which each edit blocks cut activity for a CRISPR-gRNA nuclease containing sequence complementarity to the unedited sequence. The protospacer edit weight matrix 315 is used by the cassette validator object 424 (of FIG. 4) to determine whether edits to the protospacer region are sufficient to prevent recognition of the edited sequence by the endonuclease, effectively conferring “immunity” to the expressed gRNA-CRISPR nuclease following an edit event.

The cassette scoring configuration 317 includes, but is not limited to, a PAM site cut activity threshold 318, the cassette design score function 242, the gRNA off-target activity reference sequence list 245, and the gRNA on-target cut activity model 321. The PAM site cut activity threshold 318 is the maximum allowed value for a PAM sequence, and this threshold is used by the PAM mutation comparator 434 to determine whether the PAM sequence of the modified target sequence 475 is likely to be recognized by the gRNA-nuclease complex. The cassette design score function 242 is used to generate activity scores for candidate cassettes. In one embodiment, the cassette design score function 242 can be a simple mathematical expression comprised of biological activity predictions including, but not limited to, the likelihood of gRNA on-target cut activity and off-target cut activity. All features describing biophysical characteristics, sequence composition, and alignment-based metrics generated by the candidate design feature builder 140 and an activity prediction generator 333 that predicts biological activity (e.g., formation of proteins or other substances) of a candidate cassette design 409 can be used in the cassette design score function 242. The cassette design score function 242 is a configurable parameter set by system administrators of the default configuration parameters 209 of the design library specification 110, and it is selected at run time based on the editing host organism 215 and editing kit 212 selected by the end-user.

The gRNA off-target activity reference sequence list 245 is comprised of file paths to reference sequences. This reference sequence list is input to the candidate design score calculator 150, which searches each reference sequence for regions of sequence similarity to the protospacer complementarity region of the gRNA. A subset of reference file paths are editing kit specific and determined at run time based on user-specified editing host organism 212 and editing kit 215. Editing kit 215 specific references include the editing cassette vector backbone and any other vector required for editing (e.g., a vector containing the CRISPR nuclease). Additionally, the end-user may exercise the option not to include the genome assembly, identified by the reference genome identifier 264, during the off-target search.

The gRNA on-target cut activity model 321 generates a score reflecting the likelihood that the gRNA will cut at the intended target site. In one embodiment, this model is a machine learning model trained on measured cut activity for gRNA molecules expressed from editing cassette designs produced using the cassette design engine 130 along with a feature vector comprised of biophysical characteristics (e.g. predicted secondary structure) and sequence composition (e.g., GC content) for each measured gRNA. At run time, the candidate design feature builder 140 will call a biophysical characteristic generator 324 and a sequence composition generator 327 to generate a data table for the candidate cassette designs 409. Relevant features from the data table are input into the trained gRNA on-target cut activity model 321, resulting in cut likelihood predictions. In one embodiment of the candidate cassette design engine 103, the on-target cut activity is used to generate the scored candidate cassette design library 336.

Instantiation of the candidate design feature builder 140 takes the candidate cassette designs 409 from the candidate cassette design engine 130 as input and produces an annotated candidate cassette design library 330. The cassette annotations of the annotated candidate design library 330, together with cassette metrics 418 generated by the cassette builder object 412, and the cassette scoring configuration 317 is input to the activity prediction generator 333 of the candidate design score calculator 150, resulting in a scored candidate cassette design library 336.

Features of candidate cassette design 409 include and are not limited to biophysical characteristics such as melting temperature and secondary structure stability as well as sequence composition metrics, such as length of longest homopolymer, number of unique kmers of varying length k, identity and count for particular kmers of length k, and sequence embedding or other abstract features such as those created from training a neural network. The biophysical characteristic generator 324 and sequence composition generator 327 are utilized by the candidate design feature builder 140 to develop these candidate cassette design 409 characteristics, prior to generating cassette scores for each candidate cassette design 409.

Cassette design library engine 115 generates a rank-ordered scored candidate design library 170, containing candidate cassette designs 409 scored based on expected biological activity and manufacturing requirements as discussed above. One skilled in the art using the present disclosure will recognize that there are a variety of cassette design attributes and/or predicted functionality contributing to the biological activity of a given cassette design.

By way of example and not limitation, these metrics may describe the sequence similarity between the repair template and the unedited sequence, the location of edit positions on the repair template, predictions for existence and stability of structural elements on the cassette design, sequence composition of the candidate cassette design and each component (e.g. SR, CR, HA) that makes up the cassette design.

In one embodiment the scored candidate cassette designs are sorted by a scored candidate design sort function 339, that first sorts on the final design score and then employs logic for breaking ties among cassettes with identical scores. In one embodiment, cassettes with identical designs are sorted by ancillary edit count in ascending order, with designs that impart the fewest number of ancillary edits being scored more favorably, according to one embodiment.

In one embodiment, the scored candidate cassette designs are not processed by a sort function. Instead, the best candidate design is selected using a heuristic approach comprised of a series of filtering steps. By way of example, several candidate designs have a range of design scores. All candidates with a score below a configured threshold would be filtered out of the available choices. Then all remaining candidates would be evaluated on a different attribute, like the number of ancillary edits used. All designs that confer more ancillary edits than specified by a configurable threshold would be removed from the set of choices and the remaining designs would move on to a subsequent filtering step.

FIG. 4 depicts the candidate cassette design engine 130 of the candidate design library engine 115.

The cassette design engine 130 uses a candidate cassette builder 412 to produce the candidate design library 160. The candidate cassette builder 412 is instantiated using a design specification 421 and employs a cassette assembly function 451 to produce candidate cassette designs 409 by concatenating sequences from a cassette variable region sequence set 454, cassette constant region sequences 309, and placeholder sequence 467 regions in the order specified by the cassette architecture 224 (see FIGS. 2 and 3) stored in the cassette design configuration 303.

The candidate design library 160 comprises descriptive attributes including a user-defined design library identifier 403 along with design library metrics 406 that include summary statistics, which include and are not limited to, the number of designs in the candidate cassette design list 410. Cassette design sequence 419 is comprised of a list of sequences making up a candidate cassette design 409, including the min, max, mean, and CV of the GC content, and metrics describing the sequence diversity of the candidate cassette design 409, with null values for entries for cassettes that may have failed one or more checks run by the cassette validator 424.

The design specification 421 is instantiated using several data objects defined when the design library specification 110 is parsed. In one embodiment, these objects include an edit specification 425, a target sequence description 112, the cassette design configuration 303, the cassette validator object 424 that takes validation predicates 427 as input, and a CRISPR system object 436. The edit specification 425 and the target sequence description 112 describe the sequence and location of the desired edit outcome with respect to the target sequence 267 (of FIG. 2) to be edited. The cassette validator object 424 is used to ensure that each candidate cassette 409 will function and create a minimal amount of collateral damage to the edited genomic sequence. The CRISPR system object 436 is used to determine the relative position of the SR sequence 457 and CR regions, the length of the SR sequence 457, and the PAM sequences that are recognized by the endonuclease, encapsulating these attributes which are provided to the cassette builder 412. CRISPR system object 436 enables proper identification of nuclease cut sites and configuration of the gRNA portion of each cassette design sequence 419 with enough complementarity to each target sequence to result in functional gRNA sequences.

The cassette design sequence 419 is a DNA sequence produced by the cassette assembly function 451 by concatenating several sequence components in an order specified by the cassette architecture 224 of FIGS. 2 and 3. Cassette components are classified as constant (e.g., cassette constant region sequences 309), variable (e.g., cassette variable region sequences 454), or placeholder (e.g., placeholder sequence 467) sequences. Cassette constant region sequences 309 are sequences that are defined either by system administrators or end-users and are determined at run time by the design configuration parser 120 based on the selected editing organism 212 and editing kit 215. Examples of constant region sequences include, and are not limited to, the crRNA (“CR”), restriction enzyme recognition sequences “RE,” transcription initiator sequences “TI,” and transcription terminator sequences “TT.” Examples of variable region sequences include and are not limited to the repair template homology arm “HA” and the protospacer complementarity region “SR” of the gRNA. Placeholder sequence 467 are those sequences that have a defined length at the onset of a cassette design engine 103 run, which include and are not limited to barcode sequences “BC” and amplification primer binding sites “P1” or “P2”. In one embodiment, placeholder regions will not have nucleotide sequence assignments at the termination of the cassette design engine process. Instead, these nucleotide sequences are assigned when cassette designs are selected by customers to order.

Once each component of the cassette sequence has been determined, the cassette assembly function 451 parses the cassette architecture string 224. In one embodiment, the two-letter codes for cassette components (e.g. CR, RE, TI, TT, HA, SR, BC, P1, and P2) are concatenated and delimited by the underscore symbol “_”. In one embodiment, any new component not previously used in a cassette design can be defined by the end-user during the definition of the design library specification using optional configuration settings 206 of FIG. 2. The sequence of each cassette component is included as an entry in the data table generated for the candidate design library 160.

Design of the cassette variable region sequence set 454 is a function of the cassette assembly function 451, implementing the covalent linkage between the HA sequence and the gRNA into a design, to allow for the replication vectors containing editing cassettes to be pooled and transferred to a cell population in parallel for highly efficient genome editing in multiplex. In one embodiment, the cassette variable region sequence set 454 include the protospacer complementarity region of a gRNA protospacer binding (SR) sequence 457, and the homology arm (HA) sequence 466. The length of the SR sequence 457 is set upon configuration of the CRISPR system object 436 at the onset of the cassette design engine 103 run. In contrast, the length of the HA sequence 466 is set by the design specification 421, which subtracts the lengths of all sequence components in the cassette architecture 224 from the cassette length 227, resulting in the HA sequence 466 length. Many distinct pairings of SR and HA sequences can result in the same user-specified edit sequence becoming encoded in the target sequence 267. Therefore, tens to hundreds of candidate designs (number set in the design library specification 110) are produced by the homology arm sequence generator 460, each differing in either the PAM-protospacer targeted for the cut reaction or by the ancillary edit set used to ensure highly efficient editing of the target sequence 267.

There are three steps involved in designing the HA and SR cassette components: 1) indexing the PAM-protospacer locations on the template nucleotide sequence; 2) creation of a modified target sequence 475; and 3) excising the repair template from the modified sequence 475 using homology arm slice strategy 463 specified by the design configuration 303.

In one embodiment, the homology arm sequence generator 460 employs a sequence modifier 469, which is instantiated with the design specification 421 and outputs a modified version of the input target sequence 267, a modified target sequence 475. The modified sequence 475 is generated at the same time that a PAM-protospacer site is selected as the CRISPR cut target. Thus, both the SR and HA sequences are determined by the homology arm sequence generator 460. Ultimately, the homology arm sequence generator 460 encodes the results of a slice operation on the modified sequence 475, using the homology arm slice strategy 463. As described previously, the SR sequence 457 and HA sequence 466 variable sequence regions are taken together with the cassette constant region sequences 309 and placeholder sequence 467 to produce a cassette design sequence 419 in the candidate cassette design 409.

In one embodiment, the first step of target sequence modification is the instantiation of a PAM-protospacer map object 490, which produces a PAM-protospacer index 493 all PAM-protospacer sites on the target sequence 267 that fall within the minimum and maximum allowed distance from an intended edit object 472 of multiple edit object 474. The minimum and maximum distance (measured in nucleotides) threshold are parameters encapsulated in the design specification 421. Intended edit object 472 contains one or more end-user intended edit designs defined in the edit specification list 248. Once the PAM-protospacer site index 493 exists, a PAM-protospacer site sort 496 will be applied, producing a sorted PAM-protospacer site list 499, a coordinate list sorted in order of the increasing distance between each PAM-protospacer site and the user-specified edit site. By way of example, it is possible to sort this list by distance (measured in nucleotides) between the PAM-proximal nuclease cut-site and the first nucleotide of the intended edit object 472. Similarly, it is possible to sort this list by the distance between the PAM start site and the first nucleotide of the intended edit object 472. One skilled in the art given the disclosure herein will understand that any feature on a PAM-protospacer sequence of the PAM-protospacer map object 490 and the intended edit object 472 can be used as sorting parameters.

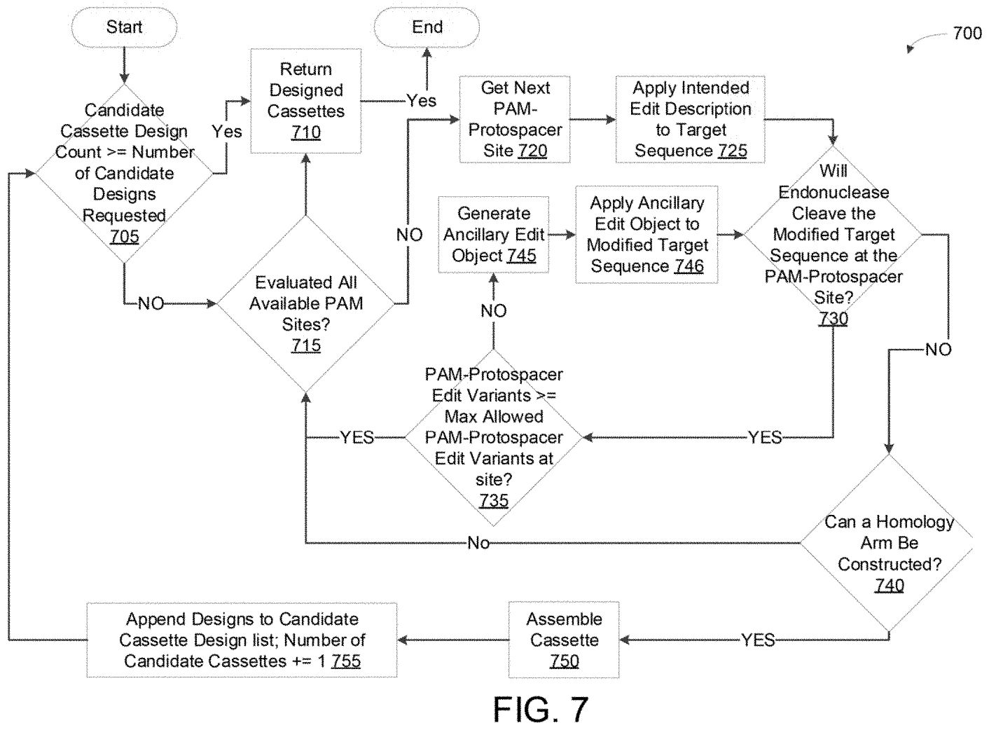

In one embodiment, following the creation of the sorted PAM-protospacer site list 499, the intended edit object 472 is used to instantiate the first instance of the multiple edit object 474. The multiple edit object 474 is then applied to the target sequence 267, defining an edited version of the target sequence 267. Subsequently, the sequence modifier 469 leverages logic in the cassette validator 424, a component of the design specification 421, to determine whether to call the ancillary edit generator 478 to build the ancillary edit object 473, an optional component of the multiple edit object 474. The cassette validator 424 will employ predicate 427 logic (described further in FIG. 7) to determine whether to create ancillary edits using a PAM-protospacer modification strategy 481 or an intervening edit strategy 484. The PAM-protospacer modification strategy 481 creates ancillary edits in order to “immunize” the modified target sequence 475 produced by the homology arm sequence generator 460, against cut activity from the CRISPR nuclease complexed with the gRNA expressed from the editing cassette. In contrast, the intervening edit strategy 484 creates ancillary edits that minimize the amount of sequence identity in the entire edit region (e.g. spanning the first to last edit coordinate) in an alignment between the unmodified target sequence 267 and the modified target sequence 475 produced by the homology arm sequence generator 460.

If the cassette validator 424 determines that ancillary edits are preferred to maximize the likelihood of generating a stable edit event, the ancillary edit generator 478 will be instructed to apply ancillary edits to the multiple edit object 474 using the appropriate strategy (e.g. 481 or 484).

Evaluation of the multiple edit object 474 applied to the modified target sequence 475 followed by the creation of additional ancillary edit objects 473 is an iterative process that terminates when either the number of ancillary edits exceeds a maximum threshold set in the design specification 421, the degree of sequence identity between the target sequence 267 and the modified sequence 475 has been minimized, or when the cassette validator 424 determines that it is unlikely the modified target sequence will be cut by the nuclease-gRNA complex.

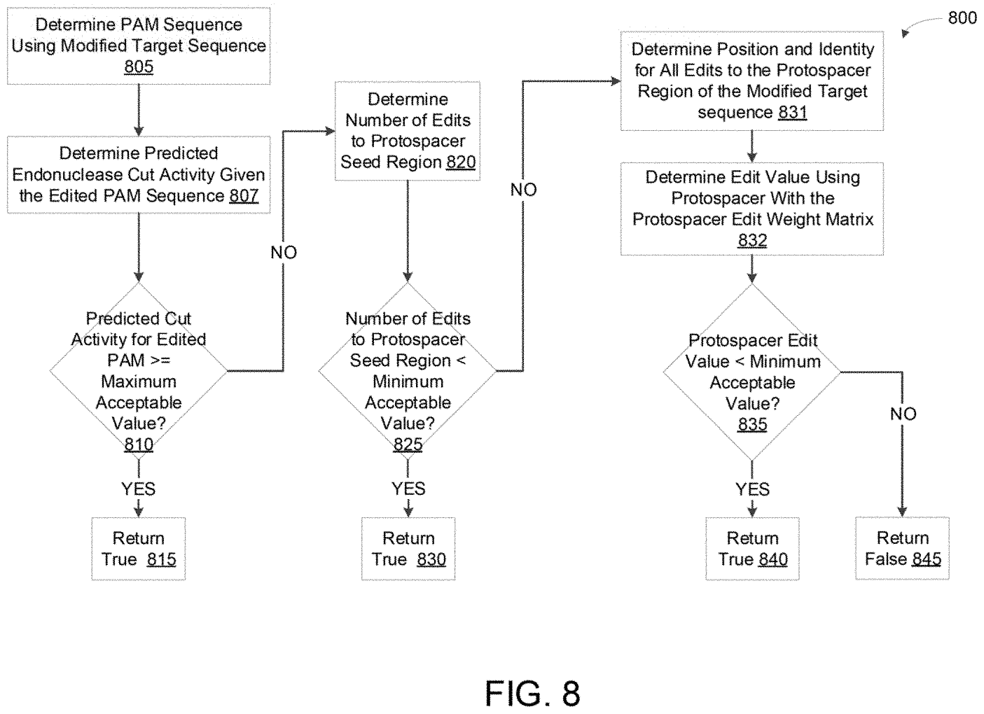

The cassette validator object 424 employs one or more sequence comparators that are responsible for evaluating one or more validation predicates 427 to determine whether an acceptable number of ancillary edits have been applied to the modified target sequence 475 and is described further below in connection with FIGS. 7 and 8. The protospacer comparator 430 of the cassette validator 424 leverages the protospacer edit weight matrix 315 of the design specification 421 to determine the number and identity of edits to the protospacer region that confer “immunity” against the cut reaction catalyzed by the expressed gRNA-CRISPR nuclease. The seed mutation comparator 433 determines whether a minimum edit threshold has been achieved in the region of the protospacer, which binds to the gRNA “seed” sequence. The gRNA “seed” sequence is defined as a region of the gRNA that must have nearly 100% sequence complementarity to the PAM-proximal subsequence of the protospacer. The length of the seed region is encapsulated in the CRISPR system object 436.

In one embodiment, care is taken by the ancillary edit generator to ensure that ancillary edits will impart a minimal impact on the biological activity of the modified target sequence 475. One of ordinary skill in the art given the teachings of the present disclosure will understand that annotations on biological sequences can be leveraged to ensure that modifications of DNA sequence can be designed in such a way as to minimize a change in biological activity. In one embodiment, the ancillary edit generator 478 accesses a codon usage table 230 and selects ancillary edits that encode synonymous codon changes to a protein-coding DNA sequence.

Synonymous codon changes ensure that the protein sequence expressed from the modified DNA sequence 475 will be identical to that of the protein sequence expressed from the unmodified target DNA sequence 267. Similarly, the activity of regulatory sequence motifs, like the Sine-Dalgarno ribosome binding site can be predicted and modifications to these sequences can be selected in order to impart a minimal change to regulatory function. A third selection process leverages a multiple sequence alignment (not shown in FIG. 4) of structured RNA regulatory elements in order to determine nucleotide changes that conserve RNA secondary structure. Finally, the end-user (or system administrator) may determine that predicting the biological impact of ancillary edits is not possible in certain DNA contexts. Under these circumstances, the end-user may choose to use multiple distinct cassette designs, differing by ancillary edit location and sequence, to impart the desired edit.

Once the modified target sequence 475 is deemed valid according to the cassette validator object 424, the homology arm sequence 466 is sliced out of the modified target sequence 475. There are homology arm slice strategies 463 for slicing the homology arm 466 from the modified target sequence 475, and this selection is indicated in the edit specification 110 sent to the cassette design engine 130. Usually, slice strategies are designed to ensure that a particular sequence element is placed at the center of the homology arm, and, by way of example, these sequences may include the PAM, PAM, and protospacer, only the protospacer, the nuclease cut site, the user-specified edit window, the ancillary edit window, or the edit window comprised of the entire set of edits introduced (e.g. ancillary and user-specified). An “edit window” is defined as the region spanning the start to the end of a particular set of edits. In another embodiment, it may be declared that a particular sequence element is placed a specified number of nucleotides from either the right or left side of the homology arm 466.

Once the final candidate cassette sequence 419 is assembled, and a unique cassette identifier 415 is assigned, a set of cassette metrics 418 are generated. Metrics capturing the location of the edit positions on the homology arm are calculated following the excision of the homology arm from the modified target sequence 475 and are included in the set of cassette metrics 418 generated by the candidate cassette builder object 412 during candidate cassette design 409. Similarly, metrics describing the sequence and location and orientation of the targeted PAM-protospacer with respect to the un-edited target sequence 267 are included in the cassette metrics 418. Other cassette metrics include, and are not limited to, the number of ancillary edits introduced during the editing reaction, unique kmer count for a given length k, and GC content.

### Example Method for Initializing Cassette Designs

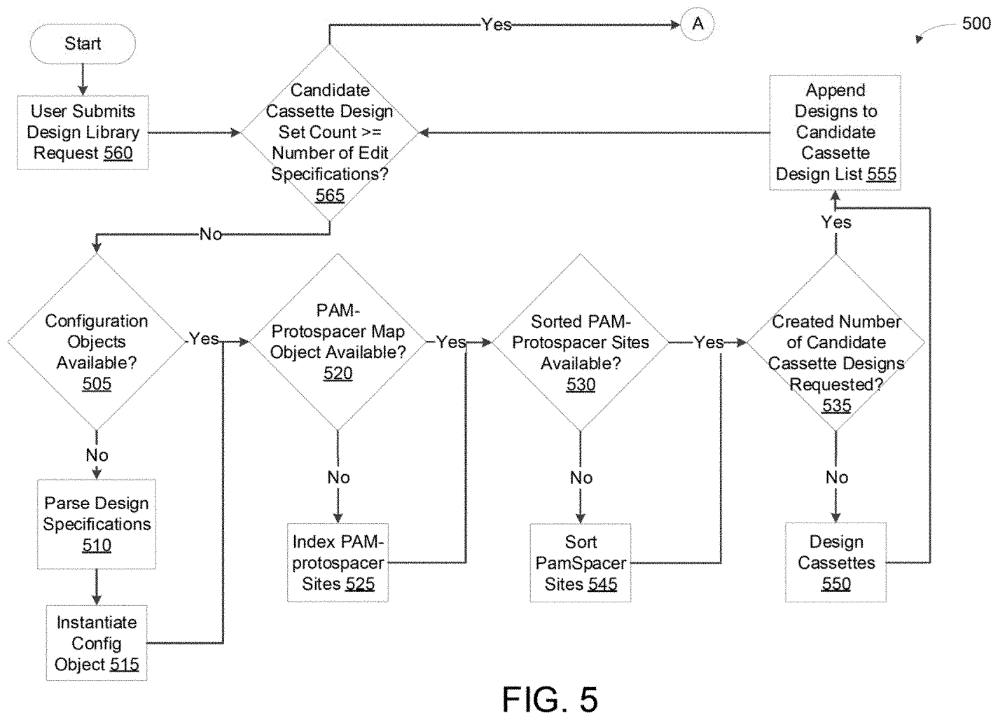

FIG. 5 depicts a method 500 for creating a library of selected candidate cassette designs 190, implementing the components of the system 100 to carry out the design library construction, according to an embodiment.

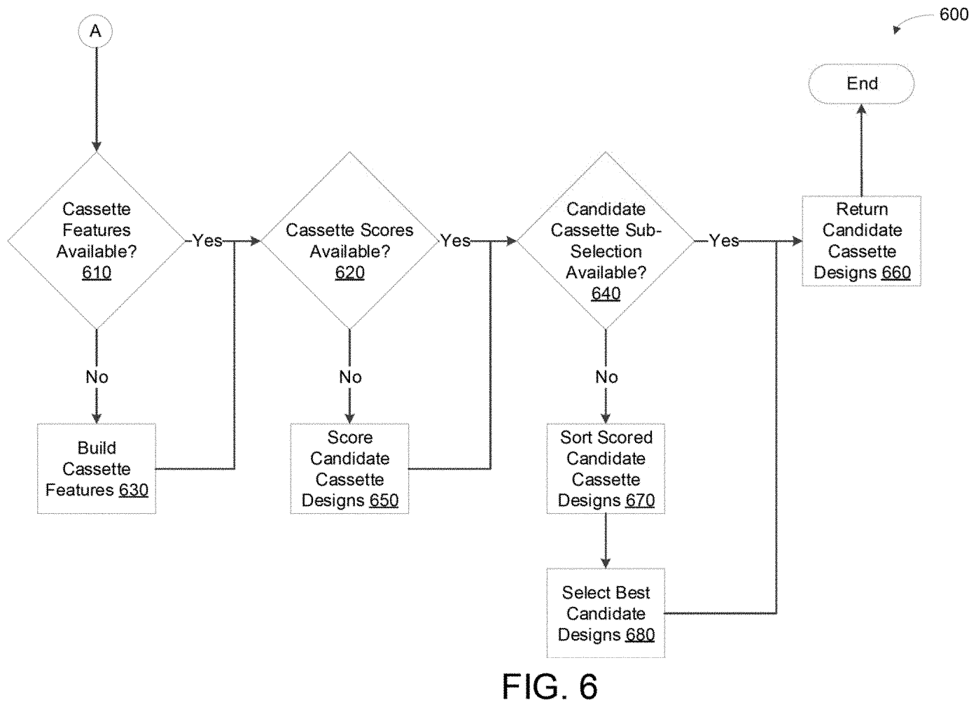

The method 500 starts with user submission of a design library request 560. At 565, method 500 evaluates whether at least one selected candidate cassette design 190 exists for each unique edit specification 251. If there is at least one, the method 500 proceeds to A, described further in FIG. 6; otherwise, the method proceeds to 505.

At 505, the method determines if there are cassette design configuration objects 303 and at least one design specification 421 available. If there is at least one available, the method proceeds to 520. Otherwise, If there are none available, the method proceeds to 510, parsing the design library specification 110 before proceeding to 515, where the cassette design configuration 303 and design specification 421 are instantiated. From the edit specification 110, the method 500 parses the cassette architecture 224, PAM activity data table 312, cassette length 227, protospacer edit weight matrix 315, and cassette constant region sequences 309, to populate the cassette design configuration 303. The design specification 421 is populated with one or more elements of the edit specification 110. The CRISPR system object 436 of design specification 421 is populated with protospacer length 439 data, PAM upstream of the protospacer 442 information, PAM-proximal nuclease cut site offset 445, and canonical PAM sequence 448 information, from The CRISPR system object 436.

Once at least one candidate cassette design configuration object 303 and design specification 421 are available, at 520, the method 500 determines if a PAM protospacer map object 490 is available for the homology arm sequence generator 460, and if so, proceeds to 530. If not, the method proceeds to 525 to generate the PAM protospacer site index 493, comprised of PAM-protospacer sites that fall within the minimum and maximum allowed distance within the target sequence 267 from the intended edit object 472 as defined by the edit description 254, parameters encapsulated in the design specification 421, before proceeding to 530.

At 530, the method 500 determines if a sorted PAM-protospacer site list 499 is available, proceeding to 535 if 530 evaluates to true. If not, at the PAM protospacer site sort 496 is called at 545 to construct the sorted PAM site list 499. The method 500 then proceeds to 535.

At 535 the method 500 determines if the method 500 has attempted to generate the number of requested candidate cassette designs 409, contained within the candidate cassette design list 410 for the given edit specification 425. If the method 500 has at least attempted to generate the number of requested candidate cassette designs 409, the cassette designs are appended to 410 at 555, otherwise, the method proceeds to 550 to create the candidate cassette designs 409, described in more detail below in connection with FIG. 7.

Once a candidate cassette design is attempted for all unique edit specifications 425, the method 500 at 565 evaluates to true, and method 500 proceeds to A, described further in FIG. 6.

FIG. 6 depicts a method for scoring cassette designs according to an embodiment. From A, the method 600 proceeds to perform a query at 610 to determine if descriptive features of the annotated candidate cassette design library 330 have been generated for each candidate design 409. If 610 evaluates to true, the method 600 proceeds to 620, otherwise the method 600 proceeds to 630, calling the candidate design feature builder 140 to generate biophysical characteristics and a sequence composition for each candidate cassette design 409.

At 620 the method 600 evaluates whether candidate cassette designs 490 have been scored, proceeding to 640 if scoring has been completed. If not, the method 600 proceeds to 650 utilizing the cassette design score calculator 150 that takes as input cassette metrics 418, sequence composition summary statistics from the sequence composition generator 327, and biophysical characteristics from biophysical characteristic generator 324 stored in the annotated candidate cassette design library 330 to generate the scored candidate cassette design library 336, and proceeds to 640.

At 640 the method 600 determines whether the set of all candidate cassette designs 409 has been sub-selected in order to return no more than the maximum allowed number of design candidates per edit specification object 251. If this determination has been made, the method 600 proceeds to 660 and returns the candidate cassette designs. If not, the method 600 proceeds to 670, calling the scored candidate design sort function 339 to sort candidate designs, resulting in the rank-ordered candidate design library 160. At 680, the method 600 calls candidate design selector 180 to sub-select design candidates from the rank-ordered candidate design library 160, and proceeds to 660. At 660 the method 600 returns the selected candidate cassette designs 190 to an end-user, ready to be synthesized on the oligomer synthesis system 195, or to the oligomer synthesis system 195.

### Example Method for Generating an Editing Cassette Design

FIG. 7 depicts a method 700 for generating editing cassette designs, according to an embodiment.

For each unique edit specification object 251, at 705 the method 700 evaluates whether the number of design candidates meets or exceeds the maximum number of allowed candidates per edit specification as defined in the cassette design configuration 303. If so, the method 700 submits the cassette designs 409 at 710 to 550 of method 500.

If not, the method proceeds to 715, and the method 700 determines if all available PAM-protospacer sites of the sorted PAM protospacer site list 499 have been evaluated. If 715 evaluates to true, the method 700 determines whether at least one candidate cassette design 409 has been created for the particular edit specification object 251. If none have been created, the method 700 generates a null cassette and proceeds to 710, providing the null cassette as the cassette design 409. Otherwise, method 700 proceeds to 720.

At 720, method 700 obtains the next PAM-protospacer site from the sorted PAM-protospacer site list 499, for evaluation. At 725 the method 700 modifies the target sequence 267 using the sequence modifier 469 to include user intended edit object 472 to produce the modified target sequence 475.

At 730, the method 700 will evaluate the modified target sequence 475 with the cassette validator object 424 to determine whether the modified target sequence is ready for processing by the homology arm slice strategy 463, detailed further in FIG. 8 below. In the event that the cassette validator object 424 determines that the modified target sequence 475 will be an equivalent substrate for the gRNA-CRISPR endonuclease as the target sequence 267, meaning that the method 100 determines that the CRISPR endonuclease will continue to cut the modified target sequence 475, the method 700 proceeds to 735. Otherwise, method 700 proceeds to 740, which evaluates to true if the homology arm slice strategy 463 is able to retrieve the homology arm sequence 466 from the modified target sequence 475. Otherwise, 740 evaluates to false and method 700 returns to 715.

At 735, method 700 determines whether the maximum allowed number of ancillary edits per PAM-protospacer has been applied to the modified target sequence 475. If 735 evaluates to true, then method 700 returns to 715, otherwise proceeding to 745. At 745, ancillary edit generator 478 invokes the PAM-protospacer modification strategy 481 for the identified PAM protospacer site, to generate an ancillary edit that is incorporated into the intended edit object 472, that will update the modified target sequence 475 to include the ancillary edit. The method 700 proceeds to 730, where the modified target sequence 475 is re-evaluated (as described in FIG. 8) to determine if the endonuclease will cleave the selected (and now edited) PAM-protospacer.

If 740 evaluates to true, then method 700 proceeds to 750, and a cassette design sequence 419 is assembled, comprising the constant region sequences 309, cassette variable region sequences 454, and placeholder sequence 467 as specified in the cassette architecture 224. The method 700 proceeds to 755, appending the recently assembled cassette design 409 to the candidate cassette design list 410, before returning to 705.

### Example Method to Determine if an Endonuclease Will Cleave a PAM Protospacer

FIG. 8 depicts an exemplary method 800 validating edits to a PAM protospacer targeted by a gRNA expressed from a gene editing cassette, according to an embodiment.

At 805, the method 800 determines the sequence of a targeted PAM site in the context of the modified target sequence 475. At 807, the PAM activity data table 312 is queried to retrieve the relative cut activity for the PAM sequence, to determine predicted nuclease cut activity.

At 810, the method 800 determines whether the relative cut activity for the PAM sequence is above the maximum allowed cut activity threshold, set in the PAM site cut activity threshold 318 of the cassette scoring configuration object 317. If 810 evaluates to true, then method 800 has determined that the gRNA expressed from the editing cassette is likely to catalyze a cut at the PAM-protospacer site in the modified target sequence 475, and a value of true is returned at 815 to 730 of method 700. Otherwise, method 800 proceeds to 820.

At 820, method 800 determines the number of single nucleotide changes encoded in the protospacer seed region within the modified target sequence 475. In one embodiment, the seed region is a subsequence of the protospacer that is proximal to the PAM and the length of the seed region is defined by the CRISPR system 436. The minimum number of edits to the seed region that are required to immunize a modified PAM-protospacer sequence against the nuclease-gRNA complex is encapsulated in the design configuration 303. At 825, the method 800 evaluates whether the number of edits to the protospacer seed region exceeds the threshold of the minimum number of edits. If 825 evaluates to true, then method 800 determines that the gRNA expressed from the editing cassette is likely to bind the target PAM-protospacer sequence of the modified target sequence 475 and at 830 returns a value of true to 730 of method 700. Otherwise, method 800 proceeds to 831.

At 831, method 800 determines the position and identity for all edits in the identified protospacer region of the modified target sequence 475 (e.g. at position 10 of the protospacer sequence, a G nucleobase is edited to an A nucleobase). Then, at 832, all edits are compared with the protospacer edit weight matrix 315 to determine the protospacer edit value. By way of example, suppose that the edited protospacer sequence has a G→A edit at position 10 and a C→A edit at position 2. It is possible that the protospacer edit weight matrix states that a G→A edit at position 10 has a weight of 0.5 and a C→A edit at position 2 has a weight of 1. If the edit value is calculated by summation, then, in this example, the protospacer edit value is 1.5. While in one embodiment of method 800 the edit value is calculated using addition of edit weights, one with ordinary skill in the art given the teaching of the present disclosure will understand that other mathematical formulas may be applied, including and not limited to, transformation to logarithmic space prior to summation, multiplication of each weight by a value equivalent to the number of edits created prior to summation, and multiplication of each positional value by a scalar followed by multiplication of all resulting values. In one embodiment, the mathematical strategy for determining the edit value is set by the design score function 242. After 832 calculates the protospacer edit value, method 800 moves to 835 which evaluates whether the protospacer edit value is less than minimum protospacer edit value is set in the design configuration object 303. If 835 evaluates to true, then method 800 at 840 returns a value of true to 730 of method 700. Otherwise, at 845 a value of false is returned to 730 of method 700.

### Example Data Illustrating Edit Efficiency Boost Using the Intervening Edit Strategy

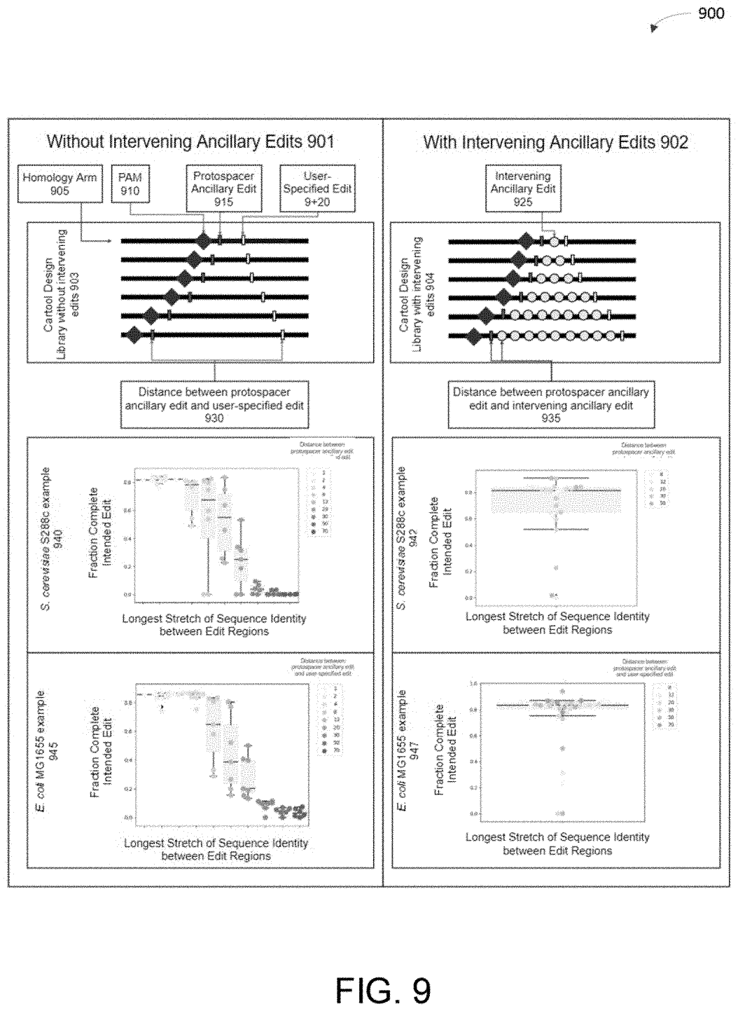

FIG. 9 shows exemplary data verifying that intervening ancillary edits increase the likelihood of a complete intended edit event when the minimum distance between the protospacer ancillary edit and the user-specified edit exceeds a maximum threshold.

In order to compare the efficacy of the intervening ancillary edit strategy 484 (of FIG. 4), two sets of selected candidate cassette designs 190 were created using system 100 and methods 500, 600, 700, and 800. Panel 901 depicts the cartoon representation of a first design library 903 that does not utilize the intervening ancillary edit strategy 484, while panel 902 illustrates a second design library 904 confers identical protospacer ancillary edits and user-specified edits as the first design library 903 and also utilizes the intervening ancillary edit strategy 484 to apply intervening ancillary edits 925 between the protospacer ancillary edit 915 and the user-specified edit 920. The cartoon illustrations of the first design library 903 and second design library 904 show a homology arm 905 (corresponding to homology arm sequence 466 of FIG. 4) of the cassette design sequences for simplicity. By way of reference, a PAM 910 of the targeted PAM-protospacer sequence is shown as a grey diamond. Each box with an “edit” label, namely protospacer ancillary edit 915, user-specified edit 920, and intervening ancillary edit 925, show regions of sequence mismatches that exist between alignment of the homology arm region from the modified target sequence 465 and the un-edited target sequence 267, located within the distance, or space, existing between the edits described above. As can be seen in the panel without intervening ancillary edits 901, the distance between the protospacer ancillary edit 915 and user-specified edit 920 can grow increasingly large, increasing the chances of an incomplete homologous recombination event and an unsuccessful editing cassette design. In the panel with intervening ancillary edits 902, the distance between edits is small, mitigating the effect of large distances between edits. The distance between protospacer ancillary edit and user-specified edit 930 and distance between protospacer ancillary edit and intervening ancillary edit 935 highlights the key difference between designs in the panels without intervening ancillary edits 903 and with intervening ancillary edits 904, which is that the length of sequence identity between edit regions in designs from 903 is greater than that of the paired designs from library 904. The existence of intervening edits 925 function to minimize the length of “intervening” homology between edit regions in designs from library 904 as compared to the paired designs from 903. As a result, there is an increased difference between the target sequence and the repair template, that benefits the process of editing a DNA sequence.

Two sets of design libraries 903 and 904 were created, targeting regions of the E. coli MG1655 genome and the S. cerevisiae S288c genome. Panels 940 and 942 illustrate the measured incorporation of all designed edits when comparing design libraries 903 and 904 created to target a region of the E. coli MG1655 genome, while, panels 945 and 947 illustrate measured edit incorporation for design libraries targeting the S. cerevisiae S288c genome. Plots 940, 942, 945, and 947 show that the fraction of complete intended edit decreases as a function of the longest stretch of sequence identity between edit regions, the distance between the protospacer ancillary edit and the user's intended edit. The longest distance between edit regions is correlated with the distance between the protospacer edit region and the user's intended edit region in plots 940 and 945, as indicated by the color gradation of plotted data. In contrast, design libraries that contain intervening edits have a constant maximum distance of 3 nucleotides between edit regions. For all distances between the protospacer ancillary edit and the user's intended edit, the fraction of observed edit events that result in a complete intended edit incorporation has a median value of ˜0.8.

Example Data Illustrating Genomic Edits from Design Libraries

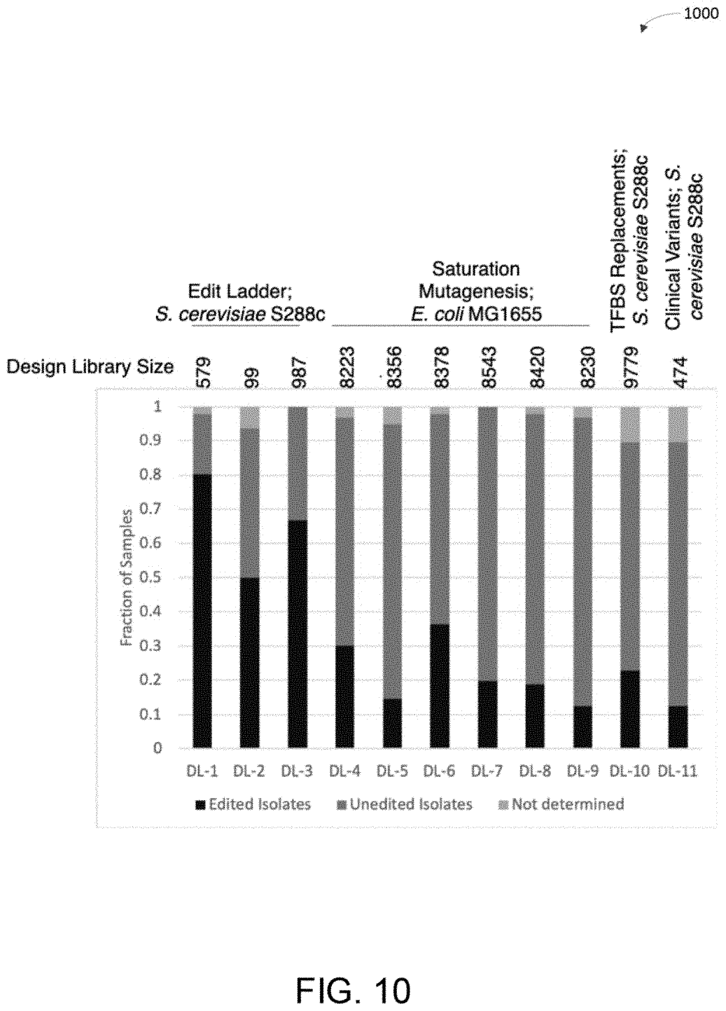

FIG. 10 depicts a stacked bar chart of edit outcomes for isolate samples taken from a population of edited cells created using design libraries built from system 100 and methods 500, 600, 700, and 800. The fraction of isolates with edited, unedited, and undetermined genomic sequences are shown with black, dark grey, and light grey bars, respectively. Unedited sequences are often the result of inactive cassette designs resulting from DNA synthesis errors, which result in lack of expression of the gRNA component of the cassette design as opposed to expressed gRNA sequences incapable of binding the CRISPR nuclease and catalyzing a DNA cut reaction (data not shown). All samples were collected as isolates in sets of 48 or 96, and often it is not possible to determine the edit outcome for all samples collected.

Design libraries are built to satisfy customer requirements, and this often means that programmed edits target several genes from a particular biosynthetic pathway, genes that give rise to the same phenotypic response when disrupted, or reconstruct variants that naturally occur in a population and have been associated with a particular disease state. By way of example, the bulk edit rate observed by sampling isolates from edited cell populations is shown for design libraries that can be placed into one of four categories: edit ladder, saturation mutagenesis, transcription factor binding site replacements (TFBS), and clinical variants. An edit ladder library encompasses design libraries that target genes that give rise to a “viable” growth phenotype when disrupted and confer a variety of edit types and edit lengths. Specifically, the edit ladder is comprised of cassettes that are evenly distributed among the edit types: swap, insertion, and deletion, and for each type of edit, designs are distributed evenly among edit lengths that span a given range (e.g. 6-75 bp). In contrast, cassette designs built to encode saturation mutagenesis are all swap edit types. Saturation mutagenesis libraries typically target a particular gene or set of genes and groups of cassette designs target the same codon position, each conferring a different codon change. Similarly, end users are often interested in changing the gene expression regulation for a particular gene or set of genes, and this can be done by editing (via swap, insertion, or replacement edit type) gene terminator sequences, promoter sequences, or transcription factor binding sites. A final example shown reflects a workflow that involves editing a non-native gene in the context of an editing host, specifically, one may edit a human gene that is expressed in a yeast cell. Using this workflow, a user may choose to create a population of edited sequences that contain sequence variants that naturally occur in the human population in order to study the effects of these variants to test efficacy of new therapeutics that may interact with genetic variants differently.

The bar chart in FIG. 10 shows three examples of edit ladder libraries that range in size from ˜100-1000 and have an average observed edit rate of 65.6% and standard deviation of 15.1%. There are six saturation mutagenesis design libraries, each with a little over 8,000 cassette designs, an average edit rate of 22%, and standard deviation of 9.3%. A single example of a transcription factor binding site replacement pool comprised of ˜10,000 cassette designs resulted in ˜23% edited isolates, and the set of ˜500 clinical variants of a human gene cloned into the S. cerevisiae S288c genome contained 12.5% edited isolates.

### Example Method for Generating an Editing Cassette Design

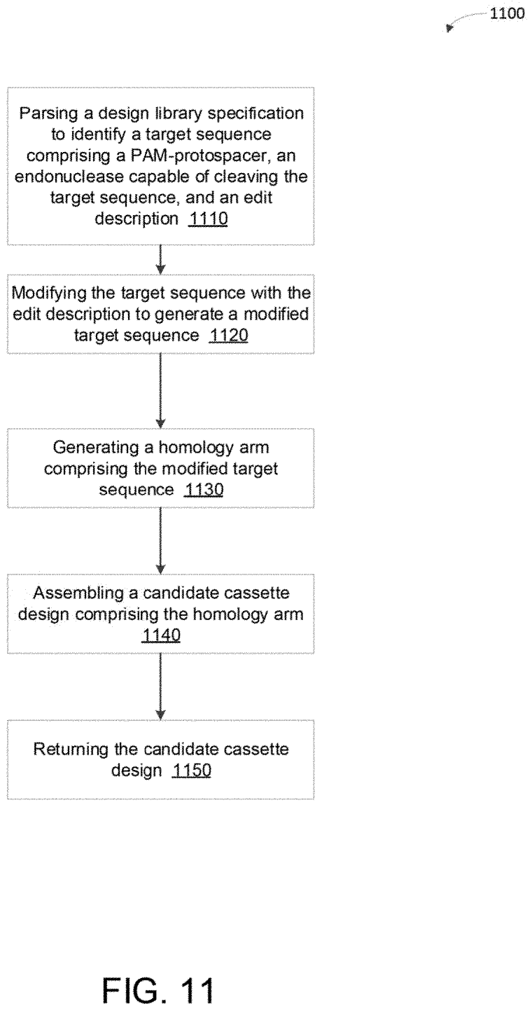

FIG. 11 depicts an exemplary method 1100 for generating an editing cassette design, according to embodiments.

At 1110, the method parses a design library specification to identify a target sequence comprising a PAM-protospacer, an endonuclease capable of cleaving the target sequence, and an edit description. In some embodiments, parsing the design library further comprises indexing a plurality of PAM-protospacers on the target sequence, the plurality of PAM-protospacers including the PAM-protospacer, and sorting the plurality of PAM-protospacers.

At 1120 the method 1100 modifies the target sequence with the edit description to generate a modified target sequence, and at 1130 the method generates a homology arm comprising the modified target sequence.

At 1140 the method 1100 assembles a candidate cassette design comprising the homology arm, and at 1150 the method returns the candidate cassette design to at least one of a user and an oligomer synthesis system.

In some embodiments the method 1100 includes determining that the endonuclease will cleave the modified target sequence substantially about the PAM-protospacer, determining that a number of edit variants applied to the PAM-protospacer are less than a maximum number of allowed edit variants, generating an ancillary edit object, and applying the ancillary edit object to the modified target sequence. In one or more embodiments, determining that the endonuclease will cleave the modified target sequence comprises one or more of determining that a prediction endonuclease cut activity score for endonuclease cut activity at the PAM-protospacer exceeds a maximum acceptable prediction score, determining that a number of edits to the PAM-protospacer is less than a minimum acceptable value, and determining that a PAM-protospacer edit value is less than a minimum acceptable value.

In some embodiments, method 1100 further comprises building cassette features based on one or more of biophysical characteristics of the candidate cassette design and sequence composition of the candidate cassette design, scoring the cassette design based on the predicted biological activity of the candidate cassette design, and selecting the candidate cassette design based on the scoring.

### Example Processing System for Generating an Editing Cassette Design

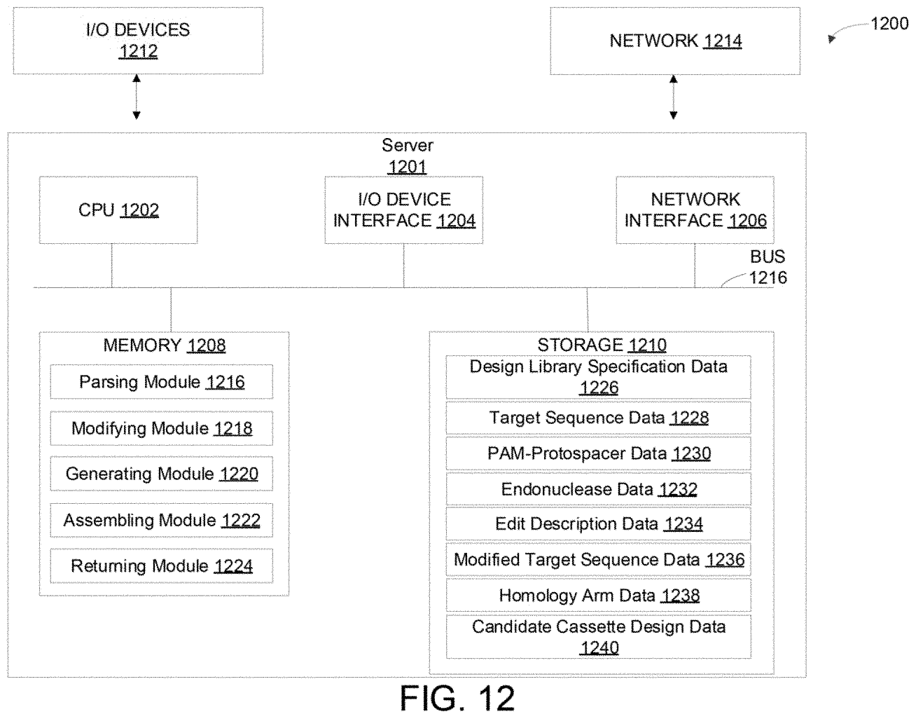

FIG. 12 depicts an exemplary processing system 1200 for generating an editing cassette design, described with respect to FIGS. 1-8, and 11.

Processing system 1200 includes server 1201, a central processing unit (CPU) 1202 connected to a data bus 1216. CPU 1202 is configured to process computer-readable instructions, e.g., stored in a memory 1208 or storage 1210, and cause the server 1201 to perform the methods described herein, for example, with respect to FIGS. 5-8. CPU 1202 is included to be representative of a single CPU, multiple CPU's, a single CPU having multiple processing cores, physical and/or virtual versions of these, and other forms of processing architecture capable of executing computer-readable instructions.

Server 1201 further includes input/output (I/O) device interface 1204, to allow server 1201 to interface with I/O devices 1212, such as, for example, keyboards, displays, mouse devices, pen input, oligomer synthesis equipment, tabletop lab equipment, and other devices that allow for interaction with server 1201. Note that server 1201 may connect with external I/O devices 1212 through physical and wireless connections.

Server 1201 further includes a network interface 1214, providing server 1201 with access to a network 1214 external to the server 1201, and thereby, external computing devices.

Server 1201 further includes memory 1208, which in this example includes a parsing module 1216, a modifying module 1218, a generating module 1220, an assembling module 1222, and a returning module 1224, and may include additional operational modules, for performing operations described in FIGS. 5-8.

Note that while shown as a single memory 1208 for simplicity, the various aspects stored in memory 1208 may be stored in different physical or virtual memories, and all accessibly by CPU 1202 via internal data connections such as bus 1216, I/O device interface 1204, and network interface 1206.

Storage 1210 further includes design library specification data 1226, which may be like the content items and operations described in FIGS. 1, 2, 5, and 11.

Storage 1210 further includes target sequence data 1228, which may be like the content items and operations described in FIGS. 2, 4-8, and 11.

Storage 1210 further includes PAM-protospacer data 1230, which may be like content items and operations described in FIGS. 1-8, and 11.

Storage 1210 further includes endonuclease data 1232, which may be like content items and operations described in FIGS. 2-8, and 11.

Storage 1210 further includes edit description data 1234, which may be like content items and operations described in FIGS. 1-8 and 11.

Storage 1210 further includes modified target sequence data 1236, which may be like content items and operations described in FIGS. 4, 7, 8, and 11.

Storage 1210 further includes homology arm data 1238, which may be like content items and operations described in FIGS. 4-8, and 11.

Storage 1240 further includes candidate cassette design data 1240, which may be like content items and operations described in FIGS. 1-8, and 11.

While not depicted in FIG. 12, other aspects may be included in storage 1210.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory), and the like. Also, “determining” may include resolving, selecting, choosing, establishing, and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A server, or other processing system used by embodiments disclosed herein, may be implemented with a bus architecture. The bus may include any number of interconnecting buses and bridges depending on the specific application of the Server and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and other circuit elements that are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the Server depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the Server to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A method for designing a gene editing cassette comprising:
receiving a design library specification comprising:
a plurality of target sequences;
a user-intended edit object comprising an edit description and a location;
a homology arm slice strategy;
a guide RNA (gRNA) endonuclease description; and
a cassette architecture comprising a variable region;
instantiating a homology arm generator object comprising a PAM-protospacer map object and a sequence modifier;
generating, with the a PAM-protospacer map object, a PAM-protospacer index of a PAM-protospacer sites for each of the plurality of target sequences, each PAM-protospacer index comprising at least one PAM-protospacer site of the respective target sequence;
generating, using the sequence modifier, a plurality of modified target sequences, each modified target sequence comprising the user-intended edit object inserted at one PAM-protospacer site of each PAM-protospacer index;
generating by the homology arm generator object, a homology arm, for each modified target sequence of the plurality of modified target sequences, based on the location of the user-intended edit object;
training a machine learning model on measured cut activity for the gRNA endonuclease description;
determining, using the trained machine learning model whether, for each homology arm, the gRNA endonuclease description will cleave the homology arm, and responsive to a determination that the gRNA endonuclease description will leave a respective one homology arm intact, copying the homology arm from the modified target sequence using the homology arm slice strategy, to generate a plurality of copied homology arms;
assembling with a cassette assembly function, a library of candidate cassette designs, for each modified target sequence comprising the copied homology arm corresponding to the respective modified target sequence, the library of candidate cassette designs comprising multiple redundant cassette designs for one or more modified target sequences having functional similarity and different cassette nucleotide sequences, each candidate cassette design defined by the cassette architecture and comprising a respective one of the copied homology arms covalently linked to a sequence encoding a gRNA, the gRNA comprising a region complementary to the target sequence in the variable region of the cassette architecture of each candidate cassette design;
modifying each candidate cassette design based on a vector array defining at least one biophysical characteristic based on the respective candidate cassette design;
generating a list of candidate cassette designs and corresponding vector arrays; and
returning the list of candidate cassette designs, wherein each candidate cassette design and corresponding vector array in the list is configured to cause an oligomer synthesis system to produce the plurality of modified target sequences.

2. (canceled)

3. (canceled)

4. The method of claim 3 wherein determining if each of the modified target sequences will be cleaved by the gRNA endonuclease description comprises one or more of:
determining that a prediction endonuclease cut activity score for endonuclease cut activity at the at least one PAM-protospacer site exceeds a maximum acceptable prediction score;
determining that a number of edits to the at least one PAM-protospacer site is less than a minimum acceptable value; and
determining that a PAM-protospacer edit value is less than a minimum acceptable value.

5. The method of claim 1 further comprising:
assembling cassette features based on one or more of elements of the vector array and sequence composition of each of the library of candidate cassette designs;
scoring each candidate cassette design, based on predicted biological activity of the candidate cassette design; and
selecting the library of candidate cassette designs, based on the scoring.

6. The method of claim 4 further comprising one of:
determining a number of edits to a protospacer seed region of each modified target sequence; and
determining a position and an identity for all edits of each modified target sequence.

7. The method of claim 6 further comprising determining a number of PAM-protospacer edits, based on a protospacer edit weight matrix, to the at least one PAM-protospacer site.

8. A non-transitory computer-readable medium comprising instructions that, when executed by a processor of a processing system, cause the processing system to:
receive a design library specification comprising:
a plurality of target sequences;
a user-intended edit object comprising an edit description and a location;
a homology arm slice strategy;
a guide RNA (qRNA) endonuclease description;
a cassette architecture comprising a variable region;
instantiate a homology arm generator object comprising a PAM-protospacer map object and a sequence modifier;
generate, with the a PAM-protospacer map object, a PAM-protospacer index of a PAM-protospacer sites for each of the plurality of target sequences, each PAM-protospacer index comprising at least one PAM-protospacer site of the respective target sequence;
generate, using the sequence modifier, a plurality of modified target sequences, each modified target sequence comprising the target sequence amended with the user-intended edit object inserted at one PAM-protospacer site of each PAM-protospacer index;
generate by the homology arm generator object, a homology arm, for each modified target sequence of the plurality of modified target sequences, based on the location of the user-intended edit object;
train a machine learning model on measured cut activity for the gRNA endonuclease description;
determine, using the trained machine learning model whether, for each homology arm, the gRNA endonuclease description will cleave the homology arm, and responsive to a determination that the gRNA endonuclease description will leave a respective one homology arm intact, copying the homology arm from the modified target sequence using the homology arm slice strategy, to generate a plurality of copied homology arms;
assemble with a cassette assembly function, a library of candidate cassette designs, for each modified target sequence comprising the copied homology arm corresponding to the respective modified target sequence, the library of candidate cassette designs comprising multiple redundant cassette designs for one or more modified target sequences having functional similarity and different cassette nucleotide sequences, each candidate cassette design defined by the cassette architecture and comprising a respective one of the copied homology arms covalently linked to a sequence encoding a gRNA, the gRNA comprising a region complementary to the target sequence in the variable region of the cassette architecture of each candidate cassette design;
modify each candidate cassette design based on a vector array, defining at least one biophysical characteristic based on the respective candidate cassette design;
generate a list of candidate cassette designs and corresponding vector arrays; and
return the list of candidate cassette designs, wherein each candidate cassette design and corresponding vector array in the list is configured to cause an oligomer synthesis system to produce the plurality of modified target sequences.

9. (canceled)

10. (canceled)

11. The non-transitory computer-readable medium of claim 8 wherein the instruction that causes the processing system to determine if each of the modified target sequences will be cleaved by the gRNA endonuclease description will cleave the modified target sequence comprises one or more of:
determine that a prediction endonuclease cut activity score for endonuclease cut activity at the at least one PAM-protospacer site exceeds a maximum acceptable prediction score;
determine that a number of edits to the at least one PAM-protospacer site is less than a minimum acceptable value; and
determine that a PAM-protospacer edit value is less than a minimum acceptable value.

12. The non-transitory computer-readable medium of claim 8 wherein the instructions further cause the processing system to:
assemble cassette features based on one or more of elements of the vector array and sequence composition of each of the library of candidate cassette designs;
score each candidate cassette design, based on predicted biological activity of the candidate cassette design; and
select the library of candidate cassette designs, based on the scoring.

13. The non-transitory computer-readable medium of claim 11 wherein the instructions further cause the processing system to perform one of:
determine a number of edits to a protospacer seed region of each modified target sequence; and
determine a position and an identity for all edits of each modified target sequence.

14. The non-transitory computer-readable medium of claim 13 wherein the instructions further cause the processing system to determine a number of PAM-protospacer edits, based on a protospacer edit weight matrix, to the at least one PAM-protospacer site.

15. A processing system comprising:
a memory comprising computer-executable instructions;
a processor configured to execute the computer-executable instructions and cause the processing system to:
receive a design library specification comprising:
a plurality of target sequences;
a user-intended edit object comprising an edit description and a location;
a homology arm slice strategy;
a guide RNA (gRNA) endonuclease description;
a cassette architecture comprising a variable region;
instantiate a homology arm generator object comprising a PAM-protospacer map object and a sequence modifier;
generate, with the a PAM-protospacer map object, a PAM-protospacer index of a PAM-protospacer sites for each of the plurality of target sequences, each PAM-protospacer index comprising at least one PAM-protospacer site of the respective target sequence;
generate, using the sequence modifier, a plurality of modified target sequences, each modified target sequence comprising the target sequence amended with the user-intended edit object inserted at one PAM-protospacer site of each PAM-protospacer index;
generate by the homology arm generator object, a homology arm, for each modified target sequence of the plurality of modified target sequences, based on the location of the user-intended edit object;
train a machine learning model on measured cut activity for the gRNA endonuclease description;
determine, using the trained machine learning model whether, for each homology arm, the gRNA endonuclease description will cleave the homology arm, and responsive to a determination that the gRNA endonuclease description will leave a respective one homology arm intact, copying the homology arm from the modified target sequence using the homology arm slice strategy, to generate a plurality of copied homology arms;
assemble with a cassette assembly function, a library of candidate cassette designs for each modified target sequence comprising the copied homology arm corresponding to the respective modified target sequence, the library of candidate cassette designs comprising multiple redundant cassette designs for one or more modified target sequences having functional similarity and different cassette nucleotide sequences, each candidate cassette design defined by the cassette architecture and comprising a respective one of the copied homology arms covalently linked to a sequence encoding a gRNA, the gRNA comprising a region complementary to the target sequence in the variable region of the cassette architecture of each candidate cassette design;
modify each candidate cassette design based on a vector array, defining at least one biophysical characteristic based on the respective candidate cassette design;
generate a list of candidate cassette designs and corresponding vector arrays; and
return the list of candidate cassette designs, wherein each candidate cassette design and corresponding vector array in the list is configured to cause an oligomer synthesis system to produce the plurality of modified target sequences.

16. (canceled)

17. (canceled)

18. The processing system of claim 15 wherein the computer-executable instructions to determine if the gRNA endonuclease description will cleave the modified target sequence comprises one or more of:
determine that a prediction endonuclease cut activity score for endonuclease cut activity at the at least one PAM-protospacer site exceeds a maximum acceptable prediction score;
determine that a number of edits to the at least one PAM-protospacer site is less than a minimum acceptable value; and
determine that a PAM-protospacer edit value is less than a minimum acceptable value.

19. The processing system of claim 15 further comprising computer-executable instructions to cause the processing system to:
assemble cassette features based on one or more of elements of the vector array and sequence composition of each of the library of candidate cassette designs;
score each candidate cassette design, based on predicted biological activity of the candidate cassette design; and
select the library of candidate cassette designs, based on the scoring.

20. The processing system of claim 18 further comprising computer-executable instructions to cause the processing system to perform one of:
determine a number of edits to a protospacer seed region of each modified target sequence; and
determine a position and an identity for all edits of each modified target sequence.
